(19) United States
(12) Patent Application Publication
(10) Pub. No.: US 2022/0293222 A1
(43) Pub. Date: Sep. 15, 2022

(54) METHODS FOR ENABLING SECURED AND PERSONALISED GENOMIC SEQUENCE ANALYSIS

(71) Applicants: CIRCAGENE LTD, London (GB); CRYPTOEXPERTS SAS, Paris (FR)

(72) Inventors: Francois Paillier, London (GB); Jackeline Palma, London (GB); Pascal Pallier, Paris (FR); Matthieu Rivain, Paris (FR); Louis Goubin, Paris (FR)

(21) Appl. No.: 17/612,501

(22) PCT Filed: May 26, 2020
(86) PCT No.: PCT/GB2020/051268

§ 371 Date: Nov. 18, 2021

(30) Foreign Application Priority Data
May 24, 2019 (GB) 1907358.4

(51) Int. Cl. G16B 50/40; G16B 20/20; G16B 30/00; H04L 9/00; G06F 21/62; G06F 21/78; G16H 10/60

(52) U.S. Cl. CPC G16B 50/40; G16B 20/20; G16B 30/00; H04L 9/008; G06F 21/6245; G06F 21/78; G16H 10/60

(57) ABSTRACT

Described herein is a secure integrated storage and analysis solution for personal genomic applications. The method guarantees data privacy whilst enabling access and ongoing analysis of genomic data when required. Described is a computer implemented homomorphic encryption method for securely producing natively encrypted sequencing data in a way that allows subsequent analysis on the encrypted data without requiring the file to be decrypted.

### RELATED APPLICATIONS

This application is a 35 U.S.C. § 371 national stage filing of International Application No. PCT/GB2020/051268, filed May 26, 2020, which claims the benefit of priority to United Kingdom Patent Application No. 1907358.4, filed May 24, 2019, the contents of each of which are herein incorporated by reference in their entirety.

### FIELD OF THE INVENTION

Described herein is an ultra-secure integrated storage and analysis solution for personal genomic applications. The method guarantees data privacy whilst enabling access and ongoing analysis of genomic data when required. The provided solution can be applied to any confidential biological information regardless of its nature or size and can also be applied to natively encrypted sequencing.

### BACKGROUND TO THE INVENTION

As methods for faster and cheaper DNA sequencing and analysis continuously emerge, the market of “Direct-to-Consumers” genetic tests is booming. The sequencing revolution paired with emergence of well characterised and clinically-actionable mutations open the way to personalised medicine and much more.

The adequate management of Genetic Data Privacy (GDP) as well as the respect of user's preferences in term of reporting requires new tools. Indeed, even though the relative standardisation of bioinformatics formats and analysis pipelines allow genetic analysts to build informative personalised genetic reports, the storage and reporting of these data requires new methods to fully respect the personal privacy preferences of each patient including in case of IT security breach or successful computer Cyber-attacks.

The danger for GDP comes also from the fact that “anonymized DNA data” might be in the future “de-anonymised” by powerful A.I. models able to integrate information within our genome and outside (including our personal social media information) and deduce the missing parts. Once our DNA sequence is disclosed, it is very difficult if not impossible to take our genetic privacy back, for the better or the worst.

Indeed, the particular nature of biological information (and especially DNA), necessitate extreme caution to properly store, protect, analyse, and communicate both raw and analysed data to the final user. By nature, the DNA sequence information can be used against the user's personal interests or for his best interests.

As such, as of 2019, a growing number of companies are marketing genetic testing kits directly to consumers in order to inform them about their genome variations for many different applications (Health, Lifestyle, Ancestry, etc). Some of these tests are sold directly to consumers (“DTC”). Subsequent communication about the genetic results occurs frequently through web applications or through websites. These applications allow online access to computing systems that extract particular genetic variations out of the total sequence data and report in a technical manner as to their relevance.

The communication of the information is focused on physicians and health care providers which are familiar with genetic variations but is difficult to understand for the end user. On top of that, in case of psychologically impactful results (such as a 60% chance of developing breast Cancer), DTC companies do not adequately adjust the test reporting to the user's preference, leading to unnecessary stress.

DTC companies such as 23andme offers web and App applications used for communicating genetic results. It consists of popular descriptions of hundreds of genetic features and associated technical reports. Although some reports are useful and actionable, many of them are either useless due to their obviousness because already known (color of eyes, teeth shapes, alcohol tolerance), non-actionable (no preventive treatment available) or difficult to understand without the help of a genetic counsellor. The focus should be on the patient himself. The focus is rather clinical, and the genetic results are thus difficult to understand for the end user. The user is offered no tools to personalize the content in this mobile application or to share or discuss certain information with its physician or any other person such as his/her Genetic counsellor.

The existing tools for interpretation and communication of next generation sequencing (NGS) raw data remain uninviting, of limited utility and too high-level for general clinicians or consumers, who do not necessarily have an extensive background in genetics and bio-informatics. Nowadays, whole genome sequencing (or whole exome sequencing) data is still predominantly used in academics and only gradually gains interest in daily clinical practice. A number of companies develop tools for analysis, annotation and interpretation of these raw sequencing data. However, existing approaches remain high-level, solely focused on experienced geneticists, often use complex user interfaces, lack flexible and responsive filtering, use limited annotation, and only few of them offer a truly actionable, affordable, secure and personalised experience to the user.

Computer files can be protected by means of encryption. Homomorphic encryption is a form of encryption that allows direct computation on “ciphertext”.

In cryptography, a ciphertext is the result of encryption performed on plaintext using a cipher type algorithm, generating a piece of encrypted information that contains a form of the original plaintext, but which is unreadable by a human or computer without the proper cipher key to decrypt it.

Homomorphic encryption is capable of performing operations on ciphertext and generating an encrypted result which, if it were decrypted, would match the result of corresponding operations that had been performed on the original piece of unencrypted plaintext. As such, homomorphic encryption can be used for secure outsourced computation, for example secure cloud computing services, and securely chaining together different services without exposing sensitive data.

In typically highly regulated industries, such as health care, homomorphic encryption can be used to enable new services by removing privacy barriers inhibiting data sharing. For example, predictive analytics in health care can be hard to utilize due to medical data privacy concerns, but if the predictive analytics service provider can operate on encrypted data instead these privacy concerns are diminished.

A cryptographic system that supports arbitrary computation on ciphertexts, in other words a system which can perform computations of any type, rather than a limited number of types of computation from a set of predefined operations, is known as fully homomorphic encryption (FHE) system.

Theoretically, a fully homomorphic encryption system can provide any desirable functionality that an unencrypted system could, running on encrypted inputs to produce an encryption of the results. As such an FHE program need never decrypt its inputs, it can be run by an untrusted party without revealing its inputs and internal state.

Cryptographic systems that support FHE thus have great practical implications in the outsourcing of private computations, for instance, in the context of cloud computing. However, relatively few FHE systems have been demonstrated to function, and those that have been have done so at great cost to security level and processing power.

For example, a useful application of FHE would be for securely querying a database. Typical database encryption leaves the database encrypted at rest, but when queries are performed the data must be decrypted in order to be parsed. A fully homomorphic encryption scheme applied to this application was demonstrated in Gahi, Youssef; Guennoun, Mouhcine; El-Khatib, Khalil (11 Dec. 2015). “A Secure Database System using Homomorphic Encryption Schemes”. However the authors noted that the scheme was both low-level and non-secure compared to regular encryption techniques, and a huge toll was taken on the performance, with operations such as a 16 bit multiplication taking approximately 24 minutes.

US2017357749 describes methods of homomorphic encryption wherein genomic data and linear prediction models are batch encoded into one or more sets of polynomials, which are then encrypted, and dot product operations are performed on the encrypted polynomials. Limiting the supported prediction models to linear models removes the need for relinearization so that the encrypted operations are not impractically slow.

In order for FHE encryption to be used for a more comprehensive, holistic analysis of bioinformatics data, the improvements described herein are desireable.

### SUMMARY OF THE INVENTION

The invention uses a computer implemented method for securely providing a user with a personally relevant analysis of biological information comprising:
a. taking a user specific electronic file containing genetic sequence information;
b. adding user specific personalised information;
c. encrypting the integrated user specific file using fully homomorphic encryption supporting non-linear prediction models, thereby combining all confidential information into a ciphertext in a way that allows subsequent analysis directly on the encrypted data without need for decrypting data to perform the computations;
d. storing the encrypted file on a user device or computation server;
e. allowing the user to compare the information in the encrypted file with external information, wherein the comparing is performed without decrypting the file; and
f. providing an individual user with user specific analysis reports of the genetic information.

The invention uses a computer implemented method for securely providing a user with a personally relevant analysis of biological information comprising:
a. taking a user specific electronic file containing a genetic sequence information;
b. adding user specific personal information;
c. encrypting the integrated user specific file using fully homomorphic encryption supporting a non-linear prediction model, thereby combining all confidential information into encrypted data in a way that allows subsequent analysis directly on said encrypted data without need for decrypting them to perform the computations;
d. storing the encrypted file on a user device or computation server;
e. performing said non-linear prediction model on the encrypted data resulting in an encrypted analysis result;
f. sending the encrypted result to a user device for decryption;
g. producing a personally relevant analysis report from the decrypted result.

The biological information may be genetic information such as nucleic acid or protein sequence data.

The term “non-linear prediction models” as used herein refers to models where data are modeled by a function which is a nonlinear combination of the model parameters, and may depend on one or more independent variables as inputs. A detailed explanation of the supported prediction models is provided below.

It is an objective of the present invention to remedy all or part of the disadvantages mentioned above. The present invention fulfils these objectives by providing methods and systems allowing for the easy and quick interpretation of a personal genorne sequence and more generally any biologically-relevant information.

The methods and systems create a powerful and secure environment allowing the user to exploit his/her own complex genorne data and facilitate the user's exploration as to the relevance of particular genome variations in an actionable way. The present invention overcomes shortcomings of the conventional art and may achieve other advantages not contemplated by the conventional software and services.

In general terms, the present invention provides a method and/or system for efficient storage and/or communication of personal aenome sequence data and/or medical information, making the relevant personal genome sequence and/or relevant medical information accessible on a mobile device or web application in an easy, secure and efficient way. The user is then free to select secure, cutting-edge methods (such as A.I. models) to analyse his own data in a way that only the user can access the analysis results.

Described is a computer implemented homomorphic encryption method for securely producing natively encrypted sequencing data in a way that allows subsequent analysis on the encrypted data without requiring the file to be decrypted.

In one embodiment, the present invention provides a method and/or system for securely providing a user with a personally relevant analysis of biological information comprising:
a. taking a user specific electronic file containing a genetic sequence information;
b. adding user specific personalised information;
c. encrypting the integrated user specific file using fully homomorphic encryption supporting non-linear prediction models, thereby combining all confidential information into a ciphertext in a way that allows subsequent analysis directly on the encrypted data without need for decrypting data to perform the computations;
d. storing the encrypted file on a user device or computation server;
e. allowing the user to compare the information in the encrypted file with known sequence information, wherein the comparing is performed without decrypting the file; and
f. providing an individual user with user specific analysis reports of the genetic information.

In one embodiment, the present invention provides a method for securely providing a user with a personally relevant analysis of biological information comprising:
a. taking a user specific electronic file containing a genetic sequence information;
b. adding user specific personal information;
c. encrypting the integrated user specific file using fully homomorphic encryption supporting a non-linear prediction model, thereby combining all confidential information into encrypted data in a way that allows subsequent analysis directly on said encrypted data without need for decrypting them to perform the computations;
d. storing the encrypted file on a user device or computation server;
e. performing said non-linear prediction model on the encrypted data resulting in an encrypted analysis result;
f. sending the encrypted result to a user device for decryption;
g. producing a personally relevant analysis report from the decrypted result.

The encryption of the file can be irreversible such that the raw data cannot be decrypted. The encrypted the can be entered using a unique user ID key, herein referred to as a GeneKey. The key allows the use to enter the encrypted the, ask specific queries of the data in the file and to generate and access reports from the file. The key is unique to the user and cannot be duplicated or replaced. The unique key allows the user and nobody else access to the user specific analysis of the genetic information. The key can be in the form of a chip card with or without contactless capacity. The key can act as both a genetic/Biometric ID card and a cryptographic key to open reports.

A unique DNA based identifier can be added to the user specific personal information at step b. The DNA based identifier can be selected from one or more of:
a. analysis of SNPs composition;
b. analysis of STRs composition;
c. analysis of Mitochondrial sequence composition; and/or
d. analysis of insertion/deletion (InDel) markers.

The SNP's or STR's can be from Chromosome Y or autosomes.

The method can contain a unique identifier determined according to DNA forensics methods added to the user specific personal information at step b, wherein
a. The forensic method being a method based on analysis of SNPs composition.
b. The forensic method being a method based on analysis of STRs composition.
c. The forensic method being a method based on analysis of Chromosome Y SNPs composition.
d. The forensic method being a method based on analysis of Chromosome Y STRs composition,
e. The forensic method being a method based on analysis of autosomes SNPs composition.
f. The forensic method being a method based on analysis of autosomes STRs composition,
g. The forensic method being a method based on analysis of Mitochondrial sequence composition.
h. The forensic method being a method based on analysis of insertion/deletion (InDel) markers,
i. The forensic method being a method combining several forensic methods such as the methods described previously.

The file can contain any information relevant to an individual. The file can contain biological sequence data including protein or nucleic acid sequence data. The data may be genetic sequence information, for example a collection of single nucleotide polymorphisms (SNP's), a whole genome sequence, a partial or exome sequence. The data may include transcriptome, proteome, metabolome, medical data or any data stored in electronic medical records or collected by quantify-self devices. The data may be an amalgamation compiled from a variety of different providers or experimental techniques, optionally including genome, transcriptome, proteome, metabolome, medical data or any data stored in Electronic Medical Records or collected by quantify-self devices.

The information may originate from a number of different providers or be sourced from two or more databases.

The user can add further information to the file. The file may therefore be supplemented with user specific personal information, for example one or more of history of illness, blood group, allergy information, birth date, location of birth, nationality, family contacts or family history of illness.

The information be automatically added to the encrypted file without requiring user input, For example the file can automatically be linked to a wearable fitness device which measures blood pressure or heart rate.

Further information can optionally be added after the file has been encrypted. Further genetic sequence or medically relevant information can be added after encryption.

The key allows access to the file to interrogate the information stored therein. The user can query the data and generate the answer to specific queries, for example disposition to future illness. The reports are also encrypted and require the user to have the key for access. The reports can be designed such that the report containing the analysed data can only be accessed once. Optionally the report containing the analysed data can only be accessed for a time limited period after creation (from few seconds to decades).

The interrogation of the encrypted file can be operated through a mobile app providing access to a variety of analysis methods. The analysis methods may be end-to-end encrypted methods. The method may be applied to the fields of health (optionally including risk prediction and predispositions analysis), nutrition (optionally including genetically optimised diet), lifestyle (optionally including daily sunlight needs or life rhythms), family history (optionally including genetic genealogy, paternity testing, forensics), and genetic-centered social interactions (optionally including genetic interest group about syndromes or Orphan diseases).

The method can be used for the analysis of an individuals medical information. Alternatively the method can be used to prove ownership of a biological organism, for example an animal or plant. For example the method can be used to authenticate origin or ownership of pets, race-horses, laboratory animals, farm animals, microorganisms, agricultural crops etc. Thus the method can be used to prove ownership, or prove the authenticity of an organism based on comparing the sequence derived from the sample with the sequence in the encrypted file.

The method can be used where genetic information is from a biological asset own by the user (whose property can be demonstrated by the user), such as without being limited to, plants, animals, synthetic biological systems or microorganisms. The biological asset can be an animal or plant used in agro-food industry, the cosmetics industry, or any other industrial domain or human activity.

The data can be analysed by a computer program. The computer program can be a classical or an Artificial Intelligence (“A.I”) program regardless of its A.I. class including without being limited to: Apriori Algorithm, Artificial Neural Networks, Collaborative Filtering, Decision Trees, Deep Learning, K Means Clustering Algorithm, Linear Regression, Logistic Regression, Naïve Bayes Classifier Algorithm, Nearest Neighbours, Random Forests, Support Vector Machine Algorithm or any method commonly described as belonging to A.I. field. The method can be a combination of programs including classical or A.I. models.

In order to further protect the information, the genetic sequence information can be encrypted at the point of sequencing a sample provided by the user.

In order to authenticate a sample, the genetic sequence information and authenticity of the sample can be encrypted at the point of origin of a sample provided by the user.

The method described includes a method wherein the content of the encrypted file combines all or part of the following elements:
a. user-specific raw data (optionally of different types, from different sources and at different level of quality),
b. user-specific analysed data; optionally including results from previous personal genomics analyses,
c. user-specific preferences data (including optionally genetic data privacy preferences, preferences in terms of type of results that must be communicated to who and how)
d. user's defined triggers optionally including file self-destruction, automated back-up options or automated transmission to a defined person;
e. a unique digital signature (either a classical digital signature or a Quantum Digital Signature).

Described is a method allowing authentication of user by digital ways during the sample collection (such as saliva sample) as well as a method to guarantee the integrity of a sample and sample shipment to the sequencing laboratory. These digital methods implement biometric authentication as well as digital tracking of the shipment and may involve the following technologies (and any combination of these technologies): GPS tracking, remote Biometric Authentication on secured software or hardware including Drones, USB Stick logger embedded in shipment and Blockchain recording of sampling and transportation events.

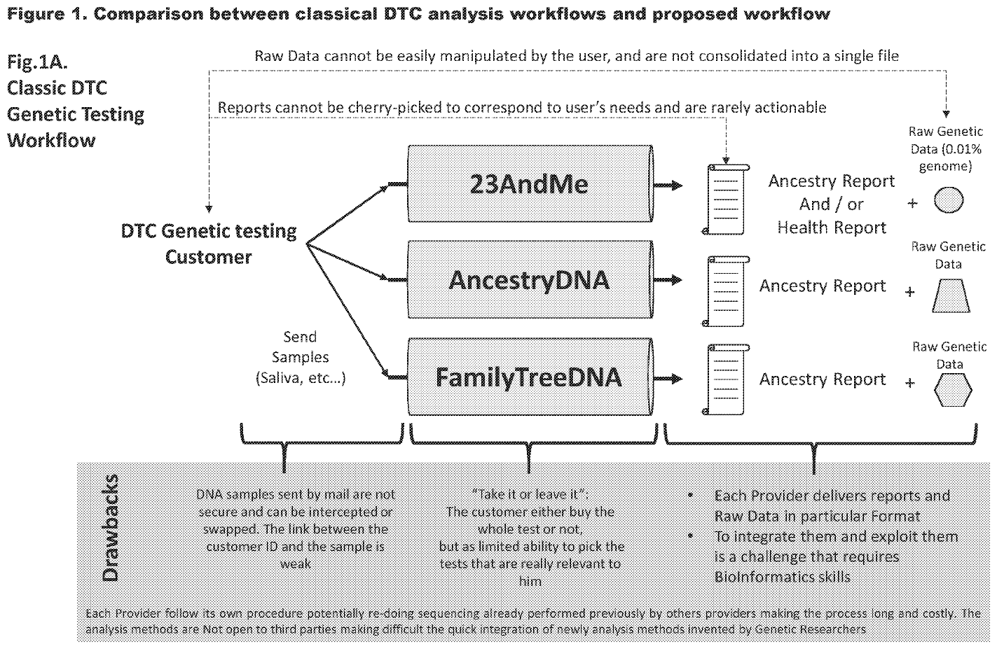

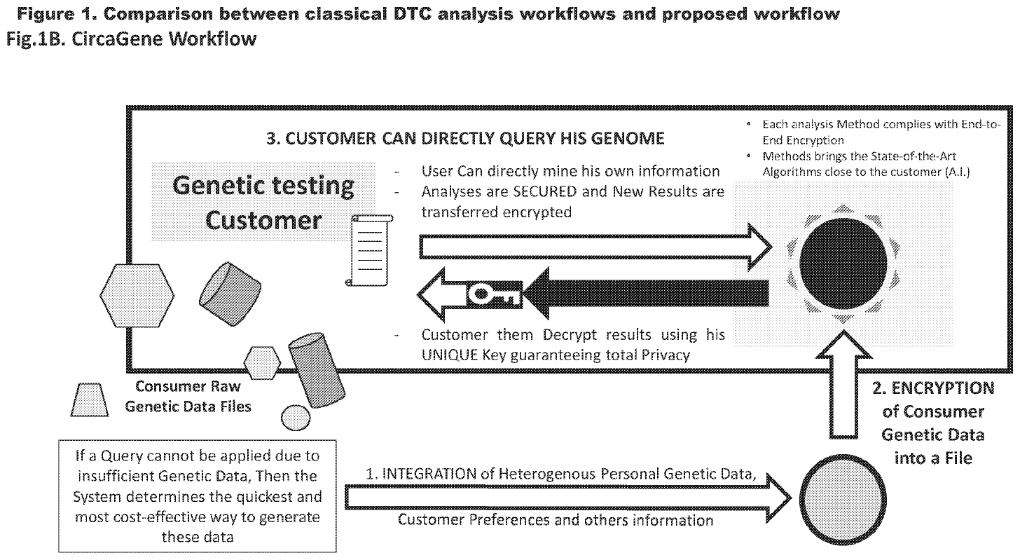

FIG. 1 Comparison between classical DTC analysis workflows (1A) and proposed workflow (1B)

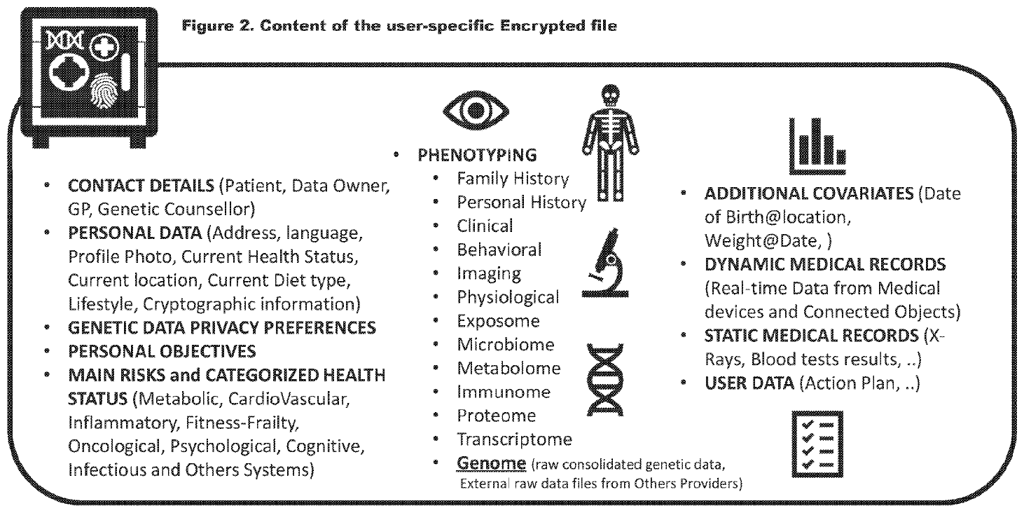

FIG. 2 Content of the user-specific Encrypted file

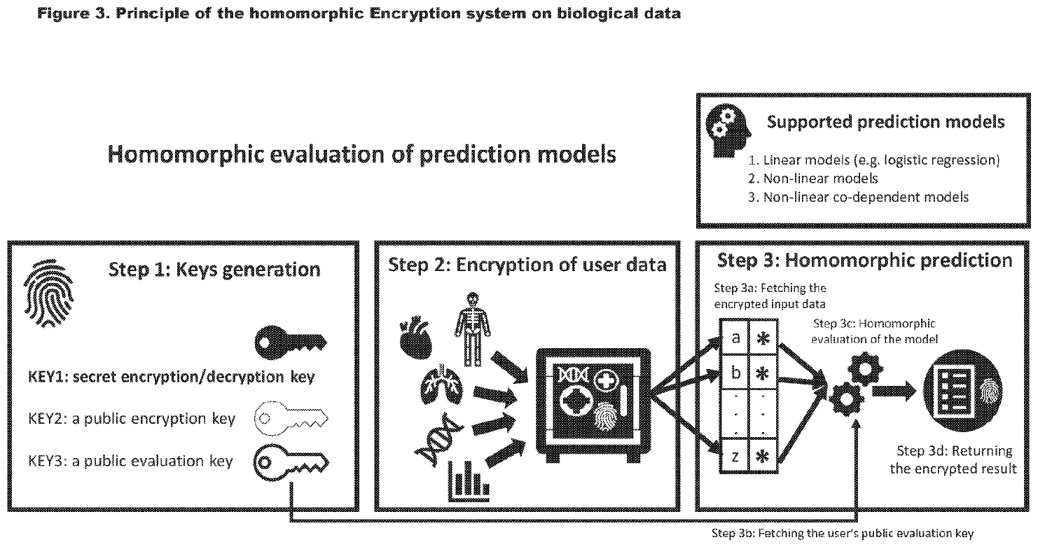

FIG. 3 Principle of the homomorphic Encryption system on biological data

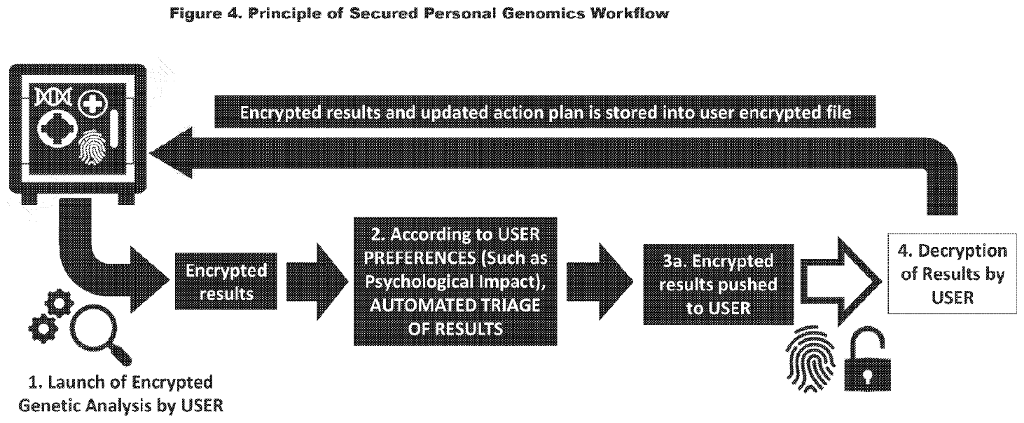

FIG. 4 Principle of Secured Personal Genomics Workflow

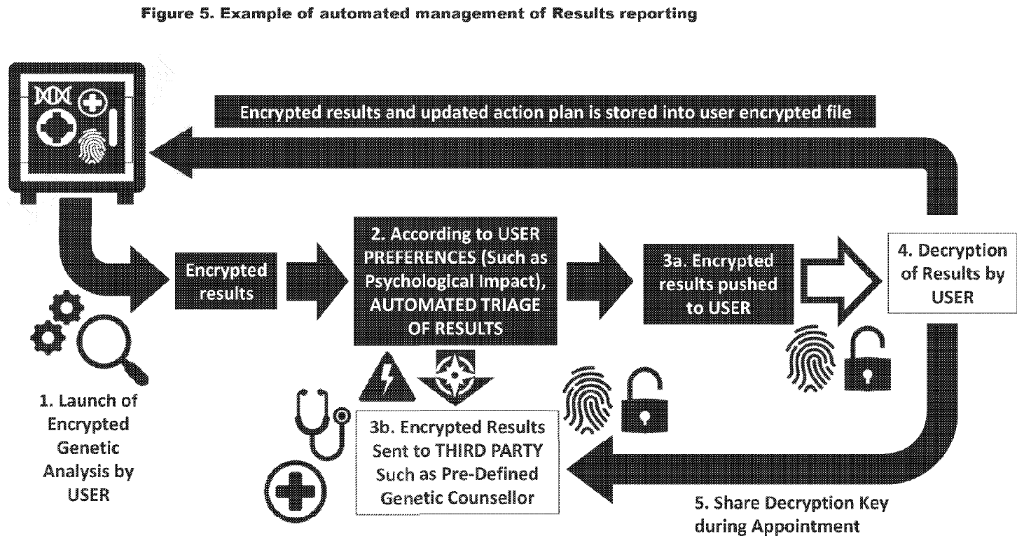

FIG. 5. Example of automated management of Results reporting

### DETAILED DESCRIPTION OF THE INVENTION

Described herein is a secure integrated storage and analysis solution for personal genomic applications. The method guarantees data privacy whilst enabling access and ongoing analysis of genomic data when required. Described is a computer implemented homomorphic encryption method for securely producing natively encrypted sequencing data in a way that allows subsequent analysis on the encrypted data without requiring the file to be decrypted.

The system allows a user to directly mine his own information. Analyses are secured and new results are transferred encrypted. Each analysis method complies with End-to-End encryption. The user decrypts results using his unique key, guaranteeing total privacy. The methods bring the state-of-the-art algorithms close to the customer, for example using A.I. As new algorithms are developed they can be applied to the encrypted data. If a query cannot be applied due to insufficient genetic data, the system determines the quickest and most cost-effective way to generate the additional data required for the query to be satisfied.

The file can be supplemented with additional data, including additional genetic data or phenotype data.

Data on the file may include one or more of:
Contact details for the patient, the data owner, medical practitioners, genetic counsellor, family members, next of kin, emergency contacts.
Personal data (address, language, profile photo, current health status, current location, current diet type, lifestyle, cryptographic information)
Genetic data privacy preferences for user and family members
Personal objectives
Main risks and categorised health status (metabolic, cardiovascular, inflammatory, fitness-frailty, ontological, psychological, cognitive, infectious)

Encryption technology described herein allows fully homomorphic encryption to support super-fast operations in the encrypted domain. The technology comes under the form of a set of software tools for use-case specifications and semi-automatic code generation.

A user's genomic data is provided in encrypted form to a service provider in order to predict a genetic trait or a risk of disease. The service provider evaluates a proprietary prediction model homomorphically on the encrypted data and returns an encrypted result without ever being able to access the genomic data in the clear. The encrypted result is then decrypted by the user—or an associated device—to view the prediction result value.

The method supports a wide class of prediction models that combine table look-ups and additive aggregation of independent gene-level contributions. Thus the invention extends far beyond logistic regression—the classical linear model for genome-wide association.

The classes of prediction models supported by the invention and the methods of their application are described below.
1. General Application of Prediction Models
1.1. Input and output

The prediction service provider is provided with a set of single nucleotide polymorphisms (or SNPs)

S=((rsid₁,x₁)(rsid₂, x₂), . . . , (rsidₙ, xₙ))

where rsidᵢ indicates the identifier of the i-th SNP and xᵢ indicates its value. For instance, when the SNPs contain a pair of nucleotide bases, each xᵢ is an ordered pair of symbols in the standard alphabet “—ACGTYRWSKMDVHBN” and can only have 136 possible values.

In addition to the set of SNPs, the prediction may require a set of covariates cov providing additional information such as age, weight, height, body mass index, ethnicity or other relevant user-specific information.

The output value of the prediction is a probability that measures the presence of a genetic trait or a health risk:

p=prediction_model(S, cov)

By applying comparison with a selected threshold probability, the result value can be made a binary value (yes or no). By apply several models in parallel, the output may also be a vector of probabilities and/or binary values.

The sets of SNPs and covariates are input into the prediction models as a single vector of value:

V=(v₁,v2, . . . vₖ)
1.2. Supported Prediction Models
1.2.1 Linear Models (e.g. Logistic Regression)

Given the input vector V=(v₁, v₂, . . . , vₖ), a linear model returns the output probability

p=f(w₀+w₁·v₁+. . . +wₖvₖ)

where the function f and all the weights w₀, w₁, . . . , wₖ are real-valued and constitute the model.

For instance, when f is chosen to be the logistic function f(t)=1/(1+e^{-t}), the model is said to be a logistic model and wₒ,wᵢ, Wₖ are called the regression coefficients. However other linear models may use different functions.

Linear models have 2 intrinsic limitations:

Limitation 1. They assume that all input variables have independent contributions in the computation of p. Indeed the contribution wᵢ·vᵢ of vᵢ is independent from all the other input variables.

Limitation 2. The contribution of an input variable vᵢ is linear in vᵢ.
1.2.2 Non-linear models

What we call here non-linear models are a generalization of linear models where

P=f(w₀+f₁(v₁)+ . . . +fₖ(vₖ))

and the coefficient w₀ as well as the functions f, f₁. . . fₖ are arbitrary and belong to the model.

Thus non-linear models escape Limitation 2. However each contribution fᵢ(vᵢ) remains independent from the other input variables, resulting in that Limitation 1 still applies.
1.2.3 Non-linear Co-Dependent Models

Non-linear co-dependent models allow each contribution to depend on arbitrary subsets of input variables.

As an example, assume that input variables in V form contiguous clusters of co-dependent variables, for instance

V=((v₁,v₁, v₂)v₃),(v₄v₅,v₆),v₇, . . .).

In this example, v₁ and v₂ form a cluster, v₃ is independent, v₄, v₅ and v₆ form another cluster, v₇ is independent, and so forth. A non-linear co-dependent model outputs

p=f(w₀+f₁₂(v₁, v₂)+f₃(v₃)+f₄₅₆(v₄,v₅,v₆)+f₇(v₇)+ . . . )

and the model parameters now include arbitrary multivariate functions.

In the general case, V is a collection of clusters (V₁, . . . , V_{q}) where each cluster V₁ is a collection of input variables Vₗ⊆{v₁, . . . , vₖ}. An input variable may belong to several clusters. The contribution of cluster Vₗ in the computation of p is fₗ(Vₗ) and the output of the model is

p=f(w₀+f₁(V₁)+ . . . +f_{q}(V_{q})).

We see that non-linear co-dependent models have no longer Limitation 1 and that
linear models [symbol] non-linear models [symbol] non-linear co-dependent models

The method as per the invention supports these 3 categories of models.
1.2.4. Why Non-Linear Co-Dependent Models Matter in Ggenomics

In linear or non-linear models, all input SNP variables have an independent effect on the final prediction result.

However, in potentially many concrete cases of genomic predictions, this is not accurate because some of the input SNPs may belong to the same gene, resulting in dependencies between the contributions of these SNPs being observed in acquired medical data.

Therefore one gets a far more accurate model by combining the SNPs belonging to the same gene together in the same cluster, and possibly adding relevant covariates to that cluster as well, so that all observed dependencies are taken into account in the model.

The particular parameters of a model (the coefficient w₀ and functions f, f₁, . . . , f_{q}) can be extracted from medical acquisitions in various ways e.g. using machine learning techniques.
2. Homomorphic Evaluation of Prediction Models

We now show how the invention allows to evaluate any non-linear co-dependent prediction model over encrypted input variables using homomorphic encryption.

Because this is the most general class of models, this description also applies—with simplifications—to linear and non-linear models.

The description that follows makes use of a generic homomorphic encryption scheme that supports:
the public encryption of integer values,
the homomorphic addition of encrypted values,
the homomorphic application of table lookups.

An encryption of an integer x is denoted [[x]].

Section 3 describes one particular reduction to practice in more detail using a particular scheme.

2.1. Step 1: Key Generation

Using the key generation procedure of the encryption scheme, the user generates 3 different cryptographic keys:
a secret encryption/decryption key sec_key,
a public encryption key enc_key,
a public evaluation key eva_key.

The user publishes enc_key so that third parties can encrypt genomic data on behalf of the user.

The user publishes eva_key so that third parties such as prediction service providers can carry out homomorphic computations over encrypted data.

The user keeps sec_key private and will use it to decrypt the encrypted prediction results.

Optionally, sec_key can also be used by the user to provide encrypted genomic data to prediction service providers.
2.2. Step 2: Encryption of User Data

User data is divided into 2 distinct categories:

1. The set of SNPs attached to the user (genomic data),

2. The set of covariates attached to the user (medical profile).
2.2.1 Encrypting the SNPs

In their standard form, the value of an SNP is an ordered pair of symbols in the alphabet “-ACGTYRWSKMDVHBN”. For non-autosomal chromosomes, or in cases of trisomy, an SNP can be composed of less or more than 2 symbols.

A convention must be adopted to encode the SNP value into an integer in an appropriate range. Typically, SNPs containing a pair of standard symbols can be encoded as an integer ranging from 1 to 136.

Alternately, the values of an SNP may be categorized into genetic variants, or groups of variants that are known to produce the same statistical effect on the medical condition of the user. In that case, the SNP value is replaced with an integer that encodes the group of variants the SNP belongs to.

In any case, if (rsidᵢ, xᵢ) denotes an SNP, we identify xᵢ with the integer-valued encoding of its value.

The above SNP is made available in encrypted form as (rsidᵢ, [[xᵢ]]) where [[xᵢ]] is a homomorphic encryption of xᵢ under the user's public encryption key enc_key.
2.2.2 Encrypting the Covariates

Covariates may be of very different nature and may rely on medical measurements in various units. By convention, the numeric representation of the j-th covariate may adopt the generic format

( [symbol] Descriptionⱼ [symbol] , cⱼ)

where (Descriptionⱼ) is a unique descriptive object (e.g. a character string or a reference to some class in an ontology) and cⱼ an integer-valued encoding of the value of the covariate. For instance,

(‘Height(cm)@2019-05-13’, 189)

may represent the user's height in centimeters at a certain date.

The above covariate is made available in encrypted form as

( [symbol] Descriptionⱼ [symbol] , [[cⱼ]])

where [[cⱼ]] is a homomorphic encryption of c₁.
2.3. Step 3: Homomorphic Prediction
2.3.1. The Homomorphic Prediction Model

The homomorphic prediction model, known by the service provider who is performing the evaluation homomorphically, is composed of:
The identifiers of all the SNPs required as input

(rsid₁, . . . , rsidₙ)
The descriptions of all covariates required as input

( [symbol] Description₁ [symbol] , . . . , [symbol] Descriptionₘ [symbol] )
The vector input clusters V₁, . . . , V_{q} and more precisely, for l=1, . . . , q
   which SNP variables i₁, . . . , iₙ_{ᵢ} are gathered in Vₗ
   which covariates j₁, . . . , jₘ_{ₗ} are gathered in Vₗ
an integer-valued weighting coefficient w₀,
an integer-valued table T_{f} that tabulates the outputs of function f over its input range,
integer-valued tables T_{f}_{₁}, . . . , T_{f}_{_{q}} where each T_{f}_{ₗ} tabulates fₗ over its input range for a given cluster.

Since the homomorphic prediction model is necessarily integer-valued, it may be obtained by approximating a continuous prediction model with an appropriate degree of precision.
2.3.2. Step 3a: Fetching the Encrypted Input Data

The prediction service provider is given the encrypted input data

[[x₁]], . . . , [[xₙ]], [[c₁]], . . . , [[cₘ]]

and for l=1, . . . , q, collects the encrypted variables belonging to cluster Vₗ:

[[Vₗ]]=( [[xᵢ_{₁}]], . . . , [[ [symbol] ]], [[cⱼ_{₁}]], . . . , [[ [symbol] ]]))
2.3.3. Step 3b: Fetching the User's Public Evaluation Key

The prediction service provider is given the user's public evaluation key eva_key.
2.3.4. Step 3c: Homomorphic Evaluation of the Model

For a given query from a user, using eva_key, the prediction service provider performs the following algorithm:

1. Initialize acc=w₀

2. For l=1 to q (2a). Perform a homomorphic table lookup with

[[V_{l]]=([[}xᵢ _{₁}]], . . . , [[ [symbol] ]], [[cⱼ_{ᵢ}]], . . . , [[ [symbol] ]])

3. on table T_{f}_{ₗ} to get the encrypted contribution, zₗ:

zₗ=[[T_{f}_{ₗ}[xᵢ_{₁}, , . . . , [symbol] , cⱼ_{₁}, . . . , [symbol] ]]]

4. of cluster Vₗ. (2b). Use homomorphic addition to aggregate over l=1 to q

acc=acc+zₗ
where acc is the accumulated value.

5. Perform a homomorphic table lookup with acc on table T_{f}. to get the encrypted prediction probability [[p]].
2.3.4. Step 3d: Returning the Encrypted Result

The encrypted prediction result [[p]] is returned to the user.
2.4 Step 4: Decryption by the User

Using the secret decryption key sec_key, the user decrypts [[p]] to get the prediction result value p in the clear.
3. Reduction to Practice

In this particular embodiment of the invention, we make use of a set of techniques based on the Torus FHE (TFHE) homomorphic encryption scheme. TFHE defines 3 distinct encryption formats TLWE, TRLWE and TRGSW with the distinct features. Only the description of TLWE is needed to show how the invention is implemented using TFHE.
TLWE Secret-Key Encryption

The plaintext is assigned a real value, μ, in the range [0,1) and is encrypted as

TLWE(μ)=(a₁, . . . , aₙ, b)

with

$$b = {\sum }_{i = 1}^{n} {a}_{i} {s}_{i} + μ + ϵmod1$$

where each aᵢ˜U_{[0,1)} is picked uniformly at random in the interval [0,1) and ϵ˜N(0, σ) is a centered Gaussian noise with variance σ².

The secret encryption-decryption key is sec_key=(s₁, . . . , sₙ) ∈ {0,1}ⁿ.

TLWE public-key encryption

Given sec_key, the encryption public key enc_key is derived by providing a vector of random encryptions of zero:

enc_key=(Z₁, . . . , Zᵣ)

where Zᵢ=TLWE(0). The public-key encryption of μ ∈ [0,1) consists in selecting random bits a₁, . . . , aᵣ ∈ {0, 1} and computing

TLWE(μ)=a₁·Z₁+ . . . +aᵣ·Zᵣ+μ mod 1.
3.1 Step 1: Key Generation

1. The user randomly selects sec_key=(s₁, . . . , sₙ) ∈ {0,1}ⁿ uniformly at random.

2. The user generates r encryptions of zero Z₁, . . . , Zᵣ and sets the encryption public key to enc_key=(Z₁, . . . , Zᵣ).

3. The user randomly generates a bootstrapping key eva_key to allow homomorphic computations by third parties.
3.2 Step 2: Encryption of User Data

To encrypt an integer variable v (an SNP value or a covariate), v is decomposed into bits v₀, . . . , vₜ₋₁ and [[v]] is defined as

$$[ [ v ] ] = ( TLWE ( \frac{{v}_{0}}{4} ) , TLWE ( \frac{{v}_{1}}{4} ) , … , TLWE ( \frac{{v}_{t - 1}}{4} ) ) .$$

3.3 Step 3: Homomorphic Prediction

Relying on the description of section 2.3.4, it is enough to provide a description of how homomorphic table lookups and homomorphic additions are performed for a single cluster of input variables.
3.3.2 Homomorphic Table Lookup

Given an encrypted cluster of integer variables

[[Vₗ]]=([[xᵢ_{₁}]], . . . , [[ [symbol] ]], [[cⱼ_{₁}]], . . . , [[ [symbol] ]]),

and since each encrypted variable is a vector of its encrypted bits under TLWE, we view [[Vₗ]] as a concatenated vector of encrypted bits under TLWE:

$$[ [ {V}_{l} ] ] = ( TLWE ( \frac{{b}_{0}}{4} ) , … , TLWE ( \frac{{b}_{t - 1}}{4} ) ) .$$

Now, TFHE provides a technique for the homomorphic evaluation of a table lookup. Let T be an arbitrary t-dimensional table of 2^{t} integer values in the range {0, . . . , 2^{d}−1}. By applying the CMux tree and gate bootstrapping techniques on the vector of encrypted bits

$$( TLWE ( \frac{{b}_{0}}{4} ) , … , TLWE ( \frac{{b}_{t - 1}}{4} ) ) ,$$

one can compute

$$( \frac{T [ {b}_{0} , … , {b}_{t - 1} ]}{{2}^{d}} )$$

where the integer d>0 is a system parameter.

In this embodiment of the invention, these techniques are used for every table lookup made necessary by the prediction model.
3.3.2 Homomorphic Addition

Since TLWE supports homomorphic additions, the current accumulated value

$$a c c = T L W E ( \frac{a}{{2}^{d}} )$$

can be updated as

$$a c c = a c c + TLWE ( \frac{T [ {b}_{0} , … , {b}_{t - 1} ]}{{2}^{d}} ) = TLW E ( \frac{a + T [ {b}_{0} , … , {b}_{t - 1} ]}{{2}^{d}} )$$

As a result of successive accumulations, the final value of the accumulator acc contains the sum

z=w₀+T_{f}_{₁}[V₁]+ . . . +T_{f}_{_{q}}[V_{q}]∈{0, . . . , 2^{d}−1}

of all contributions, namely

$$a c c = TLWE ( \frac{z}{{2}^{d}} ) .$$

In this embodiment, the function f is not applied homomorphically on acc to compute [[p]]=f([[z]]). Instead, the prediction service provider directly returns acc=[[z]] to the user together with a description of f. The function f can also be chosen once and for all as a convention between users and prediction service providers.
3.4 Step 4: Decryption by the user

Using her secret encryption-decryption key sec_key, the user

$$acc = TLWE ( \frac{z}{{z}^{a}} ) into z ∈ \{ 0 , … , {2}^{d} - 1 \} .$$

1. Decrypts 2. Applies f to z to get p=f(z).

An example is below

Among all predictive genetic tests currently available DTC, BRCA mutation testing can be considered the most actionable with proven clinical utility. Specific genetic variants in the BRCA1 and BRCA2 genes are associated with an increased risk of developing certain cancers, including breast cancer (in women and men) and ovarian cancer. These variants may also be associated with an increased risk for prostate cancer and certain other cancers. This test includes three genetic variants in the BRCA1 and BRCA2 genes that are most common in people of Ashkenazi Jewish descent.

Data relating to an individual was encrypted and the BRCA status analysed:

Case 1 BRCA1 185delAG:

| | | |
|---|---|---|
| (-;-) | 6 | 185delAG BRCA1 mutation genotype |
| (-;AG) | 6 | BRCA1 (breast cancer) 185delAG carrier |
| (AG;AG) | 0 | common in clinvar |

| | |
|---|---|
| i4000377 | 17 41276045 II □ then NON Carrier; |

Elsif DI then Carrier; Elsif DD then MUTATED= breast cancer risk
of about in Women 60%, 2% in men
Case 2

| | | |
|---|---|---|
| (-;-) | 0 | Normal |
| (-;C) | 6 | BRCA1 variant considered pathogenic for breast cancer |
| (C;C) | 6 | BRCA1 variant considered pathogenic for breast cancer |

| | |
|---|---|
| i4000378 | 17 41209083 DD □ then NON Carrier; |

Elsif DI then Carrier; Elsif II then MUTATED= breast cancer risk
of about in Women 60%, 2% in men

Or -- □ then NON Carrier;

Elsif -C then Carrier; Elsif CC then MUTATED = breast cancer risk
of about in Women 60%, 2% in men

| Case 3 | | |
|---|---|---|
| (-;-) | 6 | BRCA2 variant considered pathogenic for breast cancer |
| (-;T) | 6 | BRCA2 variant considered pathogenic for breast cancer |
| (T;T) | 0 | common/normal |

| | |
|---|---|
| i4000379 | 13 32914438 II □ then NON Carrier; |

Elsif DI then Carrier; Elsif DD then MUTATED = breast cancer risk
of about 50%, 8% in men

1. A computer implemented method for securely providing a user with a personally relevant analysis of biological information comprising:
a taking a user specific electronic file containing a genetic sequence information;
b. adding user specific personal information to form an integrated user specific file having the genetic sequence information and the user specific personal information;
c. encrypting the integrated user specific file using fully homomorphic encryption supporting a non-linear prediction model, thereby combining all confidential information into encrypted data in a way that allows subsequent analysis directly on the encrypted data without need for decrypting the encrypted data to perform the computations;
d. storing the encrypted file on a first user device or a first computation server;
e. performing the non-linear prediction model on the encrypted data resulting in an encrypted analysis result;
f. sending the encrypted result to a second user device or a second computation server for decryption;
g. producing a personally relevant analysis report from the decrypted result.

2. The method according to claim 1, wherein a unique DNA based identifier is added to the user specific personal information at step b.

3. The method according to claim 2, wherein the unique DNA based identifier is selected from one or more of:
a. analysis of single nucleotide polymorphisms (SNP's) composition;
b. analysis of Short Tandem Repeat (STR) composition;
c. analysis of Mitochondrial sequence composition; and/or
d. analysis of insertion/deletion (InDel) markers.

4. The method according to claim 3, wherein the SNP's or STR's are from Chromosome Y or autosomes.

5. The method according to claim 1, wherein the genetic sequence information is a collection of SNP's.

6. The method according to claim 1, wherein the genetic sequence information is a whole genome sequence.

7. The method according to claim 1, wherein the genetic sequence information is a partial or exome sequence.

8. The method according to claim 1 wherein the genetic sequence information is compiled from a variety of different providers or experimental techniques, optionally including transcriptome, proteome, metabolome, medical data or any data stored in Electronic Medical Records or collected by quantify-self devices.

9. The method according to claim 8, wherein the encrypted file integrates genetic user data from two or more databases.

10. The method according to claim 1, wherein the user specific personal information added includes one or more of history of illness, blood group, dietary details; blood pressure; heart rate; allergy information, birth date, location of birth, nationality, family contacts or family history of illness.

11. The method according to claim 10, wherein the personal information is updated automatically from a fitness tracker or health monitoring device,

12. The method according to claim 1, wherein the encrypted file can have further genetic sequence information added after encryption.

13. The method according to claim 1, wherein interrogation of the encrypted file is operated through a mobile app providing access to a variety of analysis methods.

14. The method according to claim 13, wherein the analysis methods are applied to one or more fields of health that includes a risk prediction and a predispositions analysis, a nutrition field that includes a genetically optimised diet, a lifestyle field that includes a daily sunlight needs or life rhythms, a family history field that includes a genetic genealogy, paternity testing, or forensics), and genetic-centered social interactions that include a genetic interest group about syndromes or Orphan diseases.

15. The method according to claim 1, wherein the genetic sequence information is from a biological asset owned by the user, such as plants, animals, synthetic biological systems or microorganisms.

16. The method according to claim 15, wherein the biological asset is an animal or a plant used in an agro-food industry, a cosmetics industry, or any other industry or human activity.

17. The method according to claim 1, wherein the genetic sequence information is encrypted at the point of sequencing a sample provided by the user.

18. The method according to claim 1, wherein the genetic sequence information and authenticity of a sample is encrypted at the point of origin of the sample as provided by the user.

19. The method according to claim 1, wherein data of the encrypted file combines all or part of the following elements:
a. user-specific raw data of different types, from different sources and at different levels of quality,
b. user-specific analysed data including results from previous personal genomics analyses,
c. user-specific preferences data including genetic data privacy preferences, preferences in terms of type of results that are communicated to who and how, and
d. a unique digital signature.
